(19) United States
(12) Patent Application Publication
(10) Pub. No.: US 2022/0003631 A1
(43) Pub. Date: Jan. 6, 2022

(54) APPARATUS AND METHOD FOR PHYSICAL LOAD TESTING

(71) Applicant: Kyur Innovative Technologies LLC, Boston, MA (US)

(72) Inventors: Naira Campbell-Kyureghyan, Boston, MA (US); Scott D. Campbell, Boston, MA (US)

(73) Assignee: Kyur Innovative Technologies LLC, Boston, MA (US)

(21) Appl. No.: 17/481,269

(22) Filed: Sep. 21, 2021

Related U.S. Application Data
(63) Continuation of application No. 15/622,421, filed on Jun. 14, 2017.
(60) Provisional application No. 62/351,801, filed on Jun. 17, 2016.

(51) Int. Cl. G01M 7/08; G09B 23/30; G09B 23/32; A41D 13/06; A41D 13/08; A61B 5/00

(52) U.S. Cl. CPC G01M 7/08; G09B 23/30; G09B 23/32; A61B 5/6829; A41D 13/081; A61B 5/6825; A41D 13/06

(57) ABSTRACT

A testing device for providing realistic response and sensor-driven data for determining the effects of physical loads on the testing devise and the effectiveness of personal protective equipment is disclosed. The testing device includes a body simulating a portion of the human body, and one or more sensors. In some embodiments, the body includes two or more body portions joined to one another. In some embodiments, the body portions may articulate with respect to one another. In some embodiments, the testing device includes an internal frame.

### CROSS-REFERENCE TO RELATED APPLICATIONS

This Application claims priority under 35 U.S.C. § 119(e) to U.S. Provisional Application No. 62/351,801, entitled “APPARATUS AND METHOD FOR PHYSICAL LOAD TESTING,” filed on Jun. 17, 2016, which is herein incorporated by reference in its entirety.

### FIELD

The disclosed embodiments are generally related to physical load testing and more particularly to apparatuses suitable for testing the effects of a physical load on a human extremity, such as on a human hand.

### BACKGROUND

Contact with objects is the third largest source of work-related injuries. For example, over 150,000 injuries occur to the hands and wrists each year, with such injuries accounting for approximately 6% of all emergency room visits in the United States. Many of these hand and wrist injuries are due to impact or crushing. Regardless of the source, hand and wrist injuries have a significant impact on the quality of life of the injured parties. Protective equipment is currently available and is intended to help mitigate the effects of contact with objects.

### SUMMARY

According to one embodiment, a testing device includes a body having a anthropomorphic hand arrangement, and one or more sensors disposed in or on the body. The one or more sensors are arranged to measure a load applied to the testing device.

According to another embodiment, a testing device includes a body having a shape of a portion of a human body, and one or more sensors disposed in or on the body. The one or more sensors arranged to measure a load applied to the testing device.

According to another embodiment, a method of testing a piece of personal protective equipment using a testing device is disclosed. The method includes applying the piece of personal protective equipment to the testing device, applying a force to the testing device, and receiving data from the one or more sensors. The testing device including a body having a shape of a portion of a human body and one or more sensors disposed in or on the body.

It should be appreciated that the foregoing concepts, and additional concepts discussed below, may be arranged in any suitable combination, as the present disclosure is not limited in this respect.

The foregoing and other aspects, embodiments, and features of the present teachings can be more fully understood from the following description in conjunction with the accompanying drawings.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows a testing device according to one embodiment;

FIG. 2 shows a testing device according to another embodiment;

FIG. 3 shows a testing device according to another embodiment;

FIG. 4 shows a testing device having an internal frame according to one embodiment;

FIGS. 5-8 are dorsal, palmar and lateral views of a testing device according to another embodiment;

FIG. 9 shows a testing device according to another embodiment;

FIG. 10 shows a testing system in which a load is applied to a testing device; and

FIG. 11 shows a testing device in which a load is applied to a testing device wearing a piece of protective equipment.

### DETAILED DESCRIPTION

Contact with objects is the third largest source of work-related injuries. For example, contact with hands, wrists, and feet can result in broken or crushed bones, or numerous other possible injuries. Although protective equipment is currently available to help mitigate the effects of contact with objects, standards regarding the effectiveness of such equipment, such as the effectiveness of a pair of gloves, do not exist.

Applicant has recognized that by providing testing devices and testing methods that allow for experimental determination of the effects of physical loads on an extremity of the human body, such as a human hand, advantages may be realized. For example, such a testing device may allow for the experimental determination of the effectiveness of personal protective equipment, such as a pair of gloves. To that end, embodiments disclosed herein include an anthropomorphic testing device, hereinafter referred to as a testing device, having a body and one or more sensors arranged to measure the effects of a physical load applied to the testing device. As will be described, the body may include one or more body portions that correspond to one or more anthropomorphic portions, such as a hand with five fingers.

In some embodiments, the testing device includes a monolithic body. For purposes herein, a monolithic body may mean that the body is formed of a single part (e.g., via 3D printing or molding). In some embodiments, the body includes one or more parts that are integrally connected to one another to form an integral body. For example, the body may be include two halves that are integrally joined together to form an anthropomorphic-shaped body.

As will be appreciated, the body may be solid or it may be hollow. In some embodiments, such a hollow body portion may be used to store one or more components of the device. For example, in embodiments in which the body is constructed of two or more parts, a user may access the inside of the body for sensor placement and/or replacement, or to allow replacement of portions of the body itself.

In some embodiments, the body includes two or more body portions that are connected to one another. As will be appreciated, the two or more body portions may be anthropomorphic portions of the body (such as the hand with five fingers). In some embodiments, the body portions are rigidly connected to each other (e.g., via a bolt, screw, or other fixed connection) such that the body portions are not movable relative to one another. In some embodiments, the body portions may be articulated with respect to one another. For example, a first body portion may be connected to a second body portion via an articulating joint. In some embodiments, the first body portion may pivot or rotate with respect to the second body portion. For example, the first body portion may be connected to the second body portion via a hinged connection. As will be appreciated, the joints may have properties chosen to mimic the corresponding joint stiffness in the human hand, or have other properties chosen in accordance with the desired test parameters.

In some embodiments, the body may include an internal frame with one or more frame portions. As will be appreciated, the frame may be monolithic or may include one or more frame portions that are connected to one another. In some embodiments, the frame portions may articulate with respect to one another (e.g., may pivot and/or rotate with respect to one another). Similar to the body, joints between the one or more frame portions may have properties chosen to mimic the corresponding joint stiffness in the human hand, or have other properties chosen in accordance with the desired test parameters.

In some embodiments, the body is constructed of a material arranged to simulate the stiffness and/or density of human tissue, or have other physical properties chosen in accordance with the desired test parameters. For example, the body may include a high biofidelity simulated hand. In some embodiments, the internal frame may be constructed of a material mimicking the stiffness of human bone, or have other physical properties chosen in accordance with the desired test parameters.

In some embodiments, the testing device includes one or more sensors arranged to measure test parameters, such as force, pressure, strain, temperature, or acceleration, which may be used to measure the physical load applied to the testing device. In some embodiments, the sensors may be positioned inside the body, such as attached to an internal frame or embedded in the material used to form the body. The sensors also may be located on the exterior of the body, or may be located both internally and on the exterior of the body. The sensors may be located at a single location or at multiple locations on the body. In some embodiments, the sensors are permanently attached to the body, although the sensors also may be removably attached to the body in some embodiments.

In some embodiments, the sensors may be arranged to measure the results of a single direction load or impact, such as a dorsal impact, a palmer impact, or a unilateral impact. The sensors also may be configured to measure a combination of two or more directions of loads or impacts.

In some embodiments, the device includes components for displaying or transmitting the measured parameters to an external or internal data acquisition, storage, or display device. In some embodiments, communication between the sensors and the external data acquisition, storage, or display devices may be accomplished through wired connections, wireless connections, or a combination of the two methods.

Alternatively, or in combination with external devices, integral devices may be provided within the body for data acquisition, storage, or display. In some embodiments, such devices may take the form of solid state memory storage device, either removable or with a manner of connection to external devices such as a USB port.

Integral devices intended to display results directly, either visual or audio devices, also may be provided in some embodiments, with or without other integral or external devices.

In some embodiments, power is provided to the sensors and/or devices. In some embodiments, the power is provided form an external device, such as via cords or wireless charging. Power to the sensors and/or devices also may be provided integrally through the use of batteries. As will be appreciated, such batteries may be rechargeable or may be disposable.

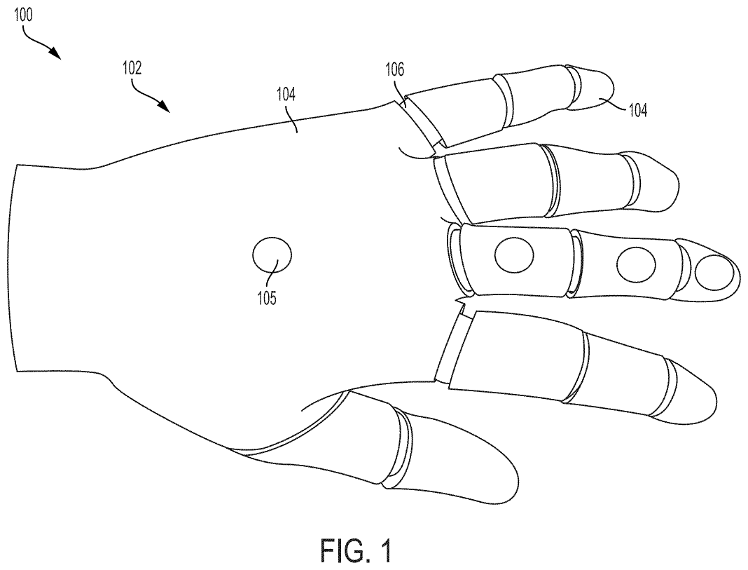

Turning now to the figures, FIG.1 illustrates a testing device 100 according to one aspect of the present disclosure. As shown in this figure, the testing device includes a body 102 having one or more body portions 104, and one or more sensors 105 to measure the effects of a physical load applied to the testing device. In some embodiments, the body 102 is arranged to be anthropometrically accurate for a portion of the human body, such as a portion of the human hand. In such embodiments, the body portions 104 may correspond to portions of the human body, such as the fingers in a human hand.

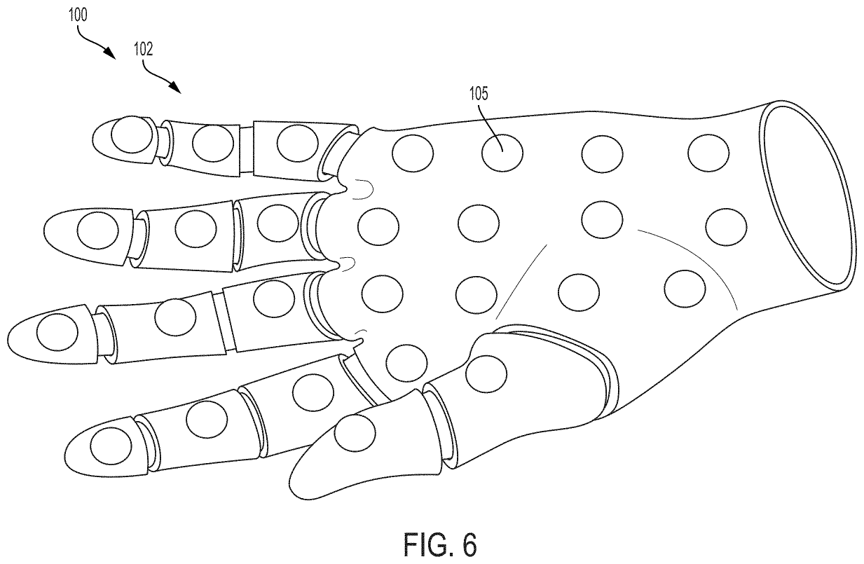

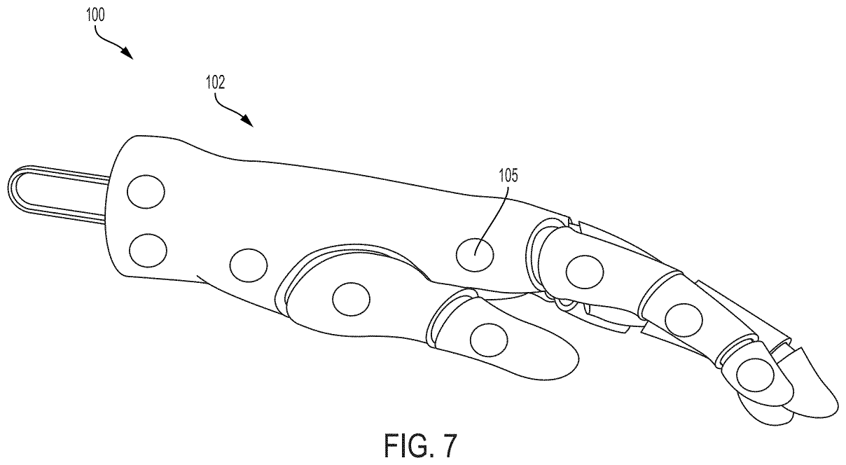

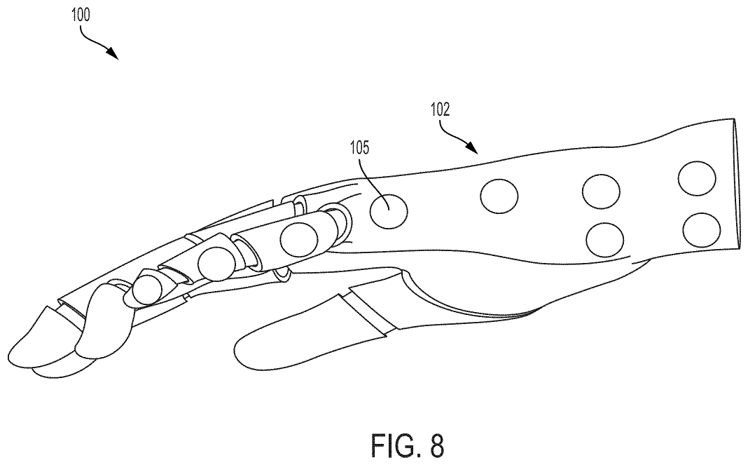

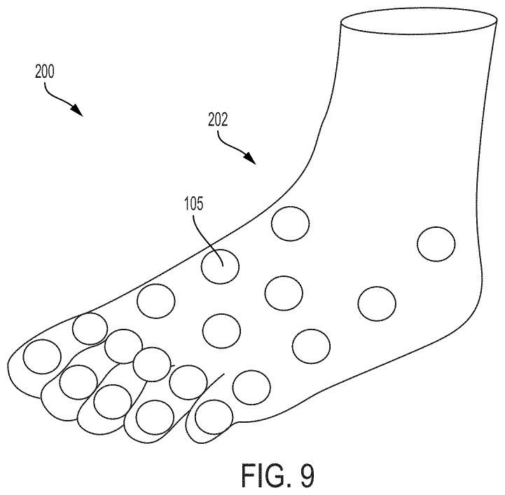

As will be a appreciated, a given body 102 may be sized for infants to adults, and from the 1^{st} to 99^{th} percentile in size. Although not shown, in some embodiments, the testing device also may include a portion of the lower arm, for example, to allow for testing of the carpal bones and/or their connection to the radius and ulna. The testing device also may include a wrist. As will be further appreciated, although the testing device is shown in the shape of a hand in FIGS. 1-8 the testing device may have other suitable shapes in other embodiments. For example, as shown in FIG. 9, a testing device 200 may be arranged to be anthropometrically accurate for a human foot. The testing device also may be arranged to be anthropometrically accurate for a human leg, chest, or another suitable portion of the body.

Turning back to FIG. 1, in some embodiments, the body 102 may be constructed of a material intended to provide high biofidelity replication of the stiffness and density of human tissue. Such a material may include a suitably-selected silicone or polyethyl alcohol, although other suitable materials may be used. Without wishing to be bound by theory, typical properties for creating high biofidelity human hands may include materials having densities between 0.9 and 1.3 g/cm³ and coefficients of restitution of 0.3-0.75, depending on the target age simulation and specific location in the hand.

In other embodiments, the body 102 may include one or more materials that differ in stiffness and density from the human hand. Such a material may be intended to provide a stable platform for sensors and/or a background for hand protective equipment. Such a material may be suitable for casting or 3D printing and includes plastics, metallic plastics, and metals.

As shown in FIG. 1, in some embodiments, the one or more body portions 104 are connected to one another via one or more joints 106. In some embodiments, the joints 106 may allow the testing device to simulate the knuckles, or other joints of the human hand, as indicated by the intended application. Such joints may be constructed of the same material as the testing device. Alternatively, the joints may be constructed of a different material, such as steel, aluminum, or plastic, and connect individual pieces of the hand test dummy body. The joints may be designed to a high level of biofidelity regarding the resulting joint stiffness, or may be constructed without consideration of the joint stiffness.

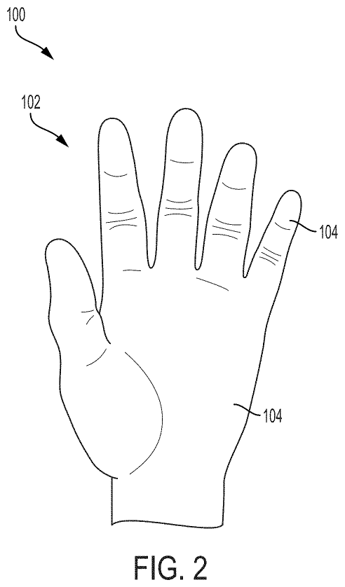

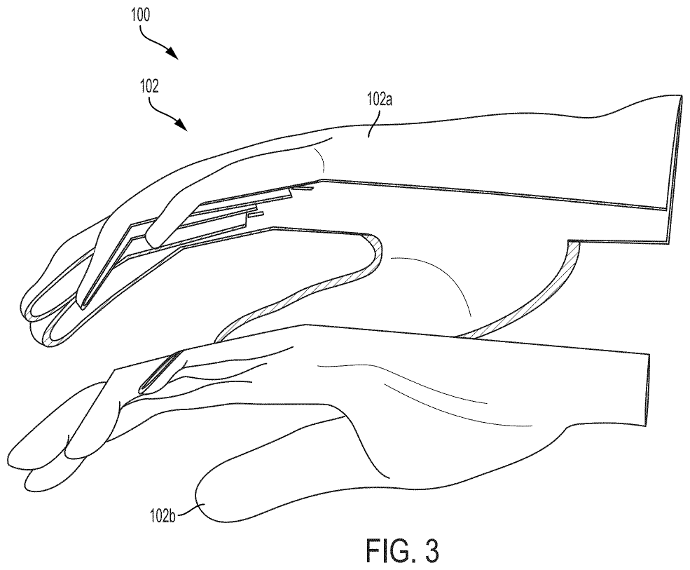

Although the testing device of FIG. 1 is shown as having articulating joints, the body also may be formed without any joints. For example, as shown in FIGS. 2-3, the device may be formed with one or more body portions 104 that are integrally connected with one another (e.g., from the same material or materials).

In some embodiments, as shown in FIG. 2, the body may be monolithic and formed of a single part (e.g., via 3D printing or molding). In other embodiments, as shown in FIG. 3, the body may be formed of two parts that are integrally connected. In such embodiments, the first and second parts 102a, 102b of the body may be permanently attached to one another. In other embodiments, the first and second parts 102a, 102b of the body may be removably attachable to one another such that the interior of the body 102 may be accessed for replacement and relocation of sensors, batteries, or communication devices. In one embodiment, the two parts 102a, 102b are created to represent the dorsal and palmer halves of the hand, respectively.

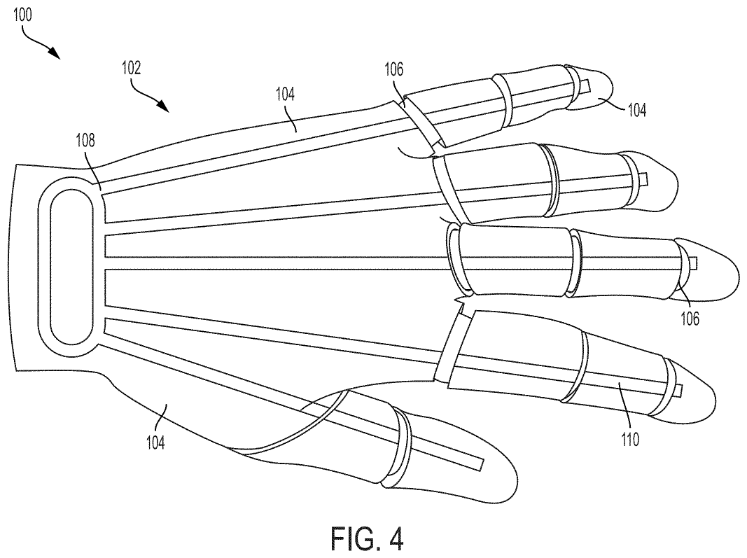

In some embodiments, as shown in FIG. 4, the testing device includes an internal frame 108. As will be appreciated, the frame 108 may include a single integrally formed part or may include one or more frame portions 110 that are joined to one another. The parts of the frame may be arranged to articulate with respect to one another (e.g., pivotably or rotatably). In some embodiments, the frame 108 is arranged to replicate the stiffness and density of human hand bones (1.5-2.0 g/cm³). Without wishing to be bound by theory, matching the frame elastic modulus to human bone in order to create a high biofidelity model may use knowledge of the intended loading direction and loading rate. For example, typical elastic modulus values for human cortical bone range between 5 and 20 GPa, depending on the loading conditions. The frame 108 also may be constructed of a material that is more rigid than human bones, such as rigid plastic, steel, or aluminum, to provide additional support to the testing device 100.

In embodiments having one or more joined frame portions, such as that shown in FIG. 4, the frame portions may be connected via joints 106 between the bones as found in the human hand, or at other locations as indicated by the intended application. The joints may further be constructed of material simulating the stiffness of human connective tissue for a high biofidelity model, or may be constructed without consideration of joint stiffness. The articulated internal frame may be used in conjunction with either a monolithic or jointed hand test model dummy body.

As will be appreciated, the internal frame also may be non-articulating. For example, the frame may be constructed as a single unit (monolithic), or may be formed via one or more frame portions that are rigidly joined together. The non-articulated internal frame may be used in conjunction with either a monolithic or a jointed hand test model dummy body.

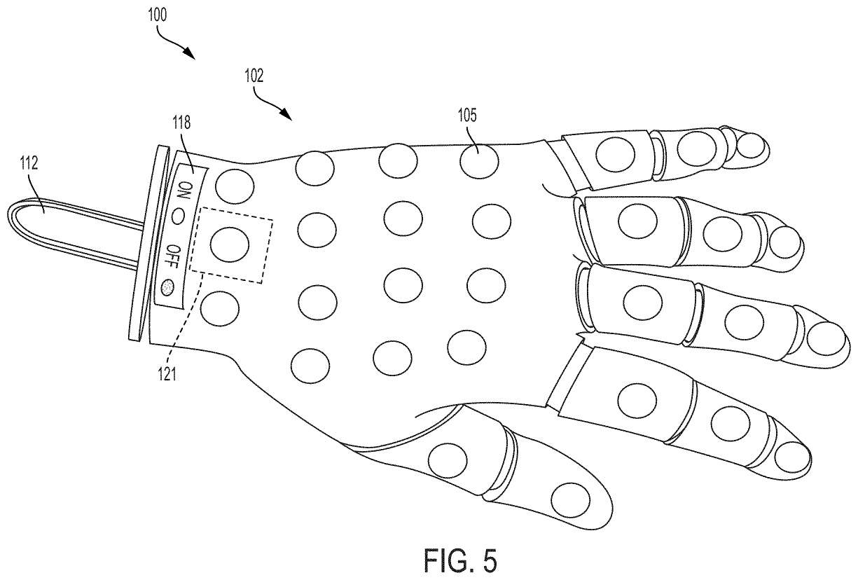

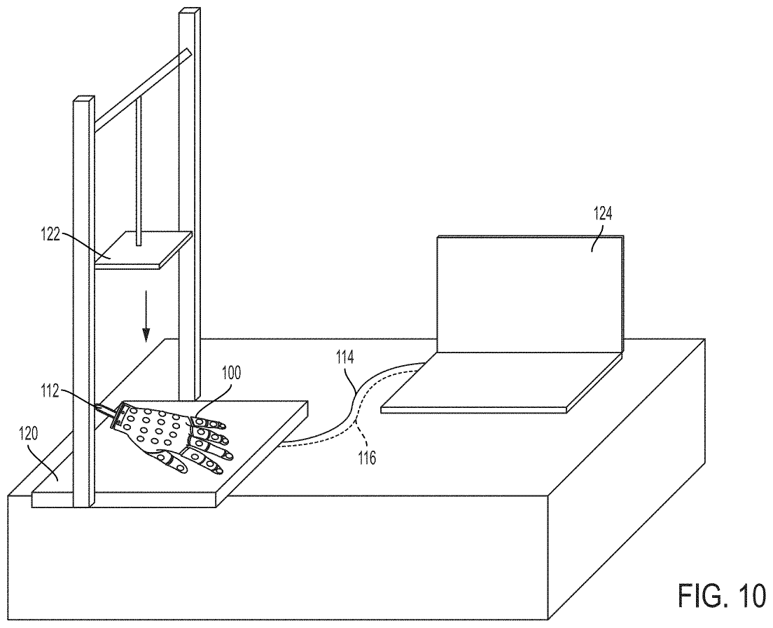

In some embodiments, as shown in FIG. 5, the testing device 100 may be equipped with a handle 112 or other position device. Such a handle may allow for ease of positioning the testing device 100 in the testing center (see FIG. 10), and may further allow for maintaining the testing device in a particular position or arrangement during testing.

Turning now to FIGS. 5-8, in some embodiments, the testing device may include one or more sensors 105 for measuring force, pressure, strain, displacement, temperature, acceleration, or other physical parameters including indications of specific loading being applied to the testing device. In some embodiments, as will be appreciated, the testing device may include only one sensor, which may be located on one of the body portions 104. In other embodiments, as shown in FIGS. 1 and 5-8, the testing device may include two or more sensors. In such embodiments, each body portion may include one or more sensor. In such embodiments, the sensors may be located on any suitable surface of the body portion.

For example, in embodiments in which the testing device is a hand, sensors may be located on each of a dorsal, palmar, and lateral side of the testing device. In some embodiments, sensors are placed at a single impact location, such as at the proximal phalange of the thumb, or at a number of locations that may be impacted simultaneously during testing, such as the proximal phalanges of the four fingers.

In some embodiments, single or multiple sensors are placed on the testing device at locations which are based on the intended type of testing to be applied. Such sensors may include devices for measuring force, pressure, strain, displacement, temperature, acceleration, or other physical parameters. As will be appreciated, each sensor need not measure the same parameter. For example, a first sensor may measure pressure while a second sensor may measure acceleration.

Although the sensors are shown as being on the exterior surface of the body, it will be appreciated that the sensors may be located inside the body, such as embedded in the material used to form the body or attached to the internal frame, or may be located in a combination thereof. As will be appreciated, the testing device therefore may allow flexibility in the positioning of the sensors through use of the multi-part body.

In embodiments in which the sensors are placed external to the testing device, the sensors may be placed on any suitable location on the body, or may be placed in specific locations as indicated by visual or mechanical indicators.

In some embodiments, the sensors are permanently attached to the body. In other embodiments, the sensors may be removably attached to the body. In such embodiments, the sensors may be moveable and replaceable. For example, one or more sensors may be attached to a first location (or first combination of locations) on the body during a first experiment and thereafter may be repositioned at a second location (or second combination of locations), different from the first location, for a second experiment.

In some embodiments, the testing device 100 includes one or more position indicators to identify a particular location or locations for a sensor or for load application (e.g., during testing). Such position indicators may be visible, such as through the use of color indicators. Such position indicators also may be mechanical, such as indentations designed to allow insertion of sensors and/or alignment of specific test equipment, or through the inclusion of positioning rigs to keep the testing device at prescribed locations.

In some embodiments, the internal frame, either articulated or monolithic, is constructed so that at a prescribed level of a parameter such as force, pressure, deformation, or strain, an externally observable indicator is activated. Such an indicator may be visual (e.g., color), audio, or some other method of indication.

In other embodiments, surface or internal indication of pressure or strain level may be visually evident upon testing. For example, an indication may be provided by applying a photoelastic material to the surface of the testing device, or embedding such a photoelastic material internally, either on an internal frame or elsewhere in the testing device.

In some embodiments, the testing device may be designed to test for the effects of physical loads in any direction including dorsal, palmar, lateral, either unilateral or bilateral, or any combination of loading directions. Sensors and/or visual indicators may be placed in multiple locations to allow for determination of output parameters from any physical load or combination of loads. In some embodiments, the testing device may apply a load in only a single direction while in other embodiments, the testing device may apply a load in two or more directions.

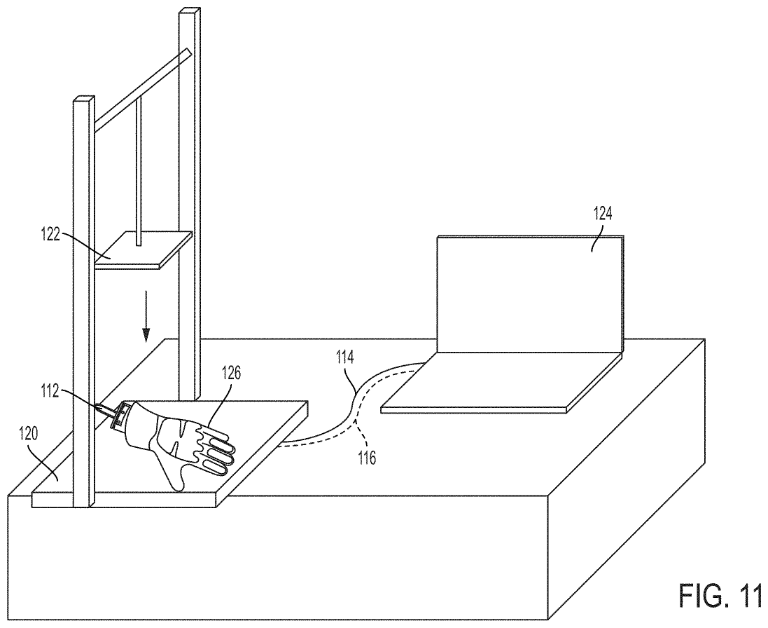

In some embodiments, the testing device is arranged to communicate the sensed data to an external data collection or display system. For example, the testing device may be arranged to communicate the data to a computer. In such embodiments, as shown in FIGS. 10-11, communication between the testing device and the external data collection or display system may be provided via a wired connection 114, such as via an analog or digital device. Communication between the testing device and the external data collection or display system also may be achieved via a wireless connection 116. Such a wireless connection may include, for example, a Bluetooth or Wi-Fi connection. In some embodiments, the testing device includes both wireless and wired communication protocols.

In some embodiments, instead of, or in addition to, communication with external data collection or display devices, the testing device may include integral data collection or display devices 118, as shown in FIG. 5. Internal data collection may take the form of flash memory, a solid state hard drive, or other device. Data transfer from the internal data collector to external devices may be either wired, such as with a USB interface, or wireless. In some embodiments, the data collection or display device 118 may include a visual indication that the device has been turned on or turned off.

In some embodiments, the integral display(s) or indicators may be included and may be analog or digital. In some embodiments, the display is a digital readout of one or more sensors. In other embodiments, the display may include a visual indicator of the sensor output, such as lights that indicate sensor reading levels through color or number of lights. Audio indicators may be used in conjunction with, or instead of, visual indicators.

In some embodiments, power to the communication and indication devices, whether wired or wireless, may be provided from an external source, or internally using batteries 121 (see FIG. 5). Such batteries may be single charge batteries or rechargeable batteries.

According to another aspect, a method of using a testing system to test a load applied to the testing device is disclosed. In some embodiments, the testing device may be used for impact and static physical load testing. In one such embodiment, as shown in FIG. 10, the testing device 100 may be placed flat on a testing surface 120 or held in a typical use position such as around a pipe. The testing device also may be held in position via the handle 112, which may be attached to the testing surface.

Next, a load may be applied to the testing device, such as by dropping a weight 122 onto the testing device. The weight 122 may be flat or be shaped as desired for the simulation, and may be guided by a track to ensure impact at the proper location on the hand test dummy. In some embodiments, the weight is dropped such that it will hit specific sensors on the testing device (such as sensors located on the dorsal surface of a hand-shaped testing device).

In some embodiments, an accelerometer may be used to determine the motion of the testing device and the impact of the applied load.

In some embodiments, a quasi-static loading may be applied using a material testing system. In such embodiments, the response of the testing device may be determined using strain gauges on an internal skeleton or force/pressure sensors located at the loading site or another location as desired. Loads may be applied in any direction to determine the response to various scenarios.

In some embodiments, cyclic loading may be applied to the testing device determine the effect of repeated loading. In addition to pressure/force or strain gauges, temperature may be measured internally to estimate the energy imparted to the hand or to evaluate heat buildup.

As will be appreciated, combinations of the above-noted loads and types may be applied during an experiment to determine the effects of the load on the testing device.

The sensed data may be communicated to an external data collection device, such as a computer 124. Such communication may be a wired 114 or wireless 116 communication. The data may be analyzed via a program on the computer.

In some embodiments, the effects of the applied load are determined by visual inspection of the testing device. For example, after application of the load, the testing device may be examined to see if the internal frame, simulating a person's bones, has been fractured and/or crushed. As another example, the exterior surface of the body of the testing device, simulating a person's tissue, has been cut and/or deformed.

In some embodiments, as shown in FIG. 11, prior to applying a load, a piece of protective equipment, such as a glove 126 or other piece of apparel, may be placed on the testing device. As will be appreciated, in embodiments in which the testing devices is arranged to be anthropometrically accurate for a human foot, a sock or boot may be placed on the testing device. Next, the load may be applied and the sensor may measure one or more test parameters. The sensor output data may then be communicated to the computer 124, where the data is received and analyzed to determine the effectiveness of the piece of equipment.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The terms “attached” and “connected” are used broadly and encompass both direct and indirect attaching and connecting. Further, “connected” is not restricted to physical or mechanical connections, and can include electrical connections, whether direct or indirect. Also, electronic communications and notifications may be performed using any known means, such as direct connections and wireless connections.

It should also be noted that a plurality of hardware and software based devices, as well as a plurality of different structural components may be used to implement the invention. In addition, it should be understood that embodiments of the invention may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software (e. g., stored on non-transitory computer-readable medium) executable by one or more processors. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

What is claimed is:

1-39. (canceled)

40. A testing system comprising:
a hand-shaped testing device including a body and an internal frame disposed in the body;
one or more sensors located on a dorsal side of the body, the one or more sensors arranged to measure a load applied to the hand-shaped testing device;
a protective glove positioned over the hand-shaped testing device;
a testing surface on which the hand-shaped testing device and protective glove are placed; and
a load applicator configured to apply a load to the hand-shaped testing device.

41. The testing device of claim 40 wherein the internal frame includes two or more frame portions.

42. The testing system of claim 41, wherein the two or more frame portions are connected via joints between the frame portions.

43. The testing device of claim 41, wherein the frame has an elastic modulus of between 5 and 20 GPa.

44. The testing system of claim 40, wherein the one or more sensors are configured to produce sensor output data, and further comprising a computer configured to analyze the sensor output data to determine an effectiveness of the protective glove.

45. The testing system of claim 40, wherein the body comprises first and second body portions, and the first body portion is connected to the second body portion with an articulating joint.

46. The testing system of claim 40, wherein the one or more sensors are attached to the frame.

47. The testing system of claim 40, wherein the one or more sensors are configured to measure a combination of two or more directions of loads.

48. The testing system of claim 40, wherein the load applicator comprises a weight configured to be dropped on the testing device.

49. The testing system of claim 40, further comprising a track, wherein the weight is guided by the track.

50. A testing system comprising:
a foot-shaped testing device including a body and an internal frame disposed in the body;
one or more sensors located on a dorsal side of the body, the one or more sensors arranged to measure a load applied to the foot-shaped testing device;
a protective foot covering positioned over the foot-shaped testing device;
a testing surface on which the foot-shaped testing device and the protective foot covering are placed; and
a load applicator configured to apply a load to the foot-shaped testing device.

51. The testing device of claim 50 wherein the internal frame has two or more frame portions.

52. The testing system of claim 51, wherein the two or more frame portions are connected via joints between the frame portions.

53. The testing device of claim 51, wherein the frame has an elastic modulus of between 5 and 20 GPa.

54. The testing system of claim 50 wherein the one or more sensors are configured to produce sensor output data, and further comprising a computer configured to analyze the sensor output data to determine the amount of protection provided by the impact effectiveness of the protective foot covering.

55. The testing system of claim 50, wherein the body comprises first and second body portions, and the first body portion is connected to the second body portion with an articulating joint.

56. The testing system of claim 50, wherein the one or more sensors are attached to the frame.

57. The testing system of claim 50, wherein the one or more sensors are configured to measure a combination of two or more directions of loads.

58. The testing system of claim 50, wherein the load applicator comprises a weight configured to be dropped on the testing device.

59. The testing system of claim 50, further comprising a track, wherein the weight is guided by the track.

60. The testing system of claim 50, wherein the protective foot covering comprises a boot.
